(19) United States
(12) Patent Application Publication
(10) Pub. No.: US 2004/0161454 A1
(43) Pub. Date: Aug. 19, 2004

(54) Active ingredient-containing matrix patches

(75) Inventors: Michael Schink, Hamburg (DE); Ulrich Kohler, Hamburg (DE); Holger Kartheus, Hamburg (DE); Peter Philipp, Oststeinbek (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG, Hamburg (DE)

(21) Appl. No.: 10/700,932

(22) Filed: Nov. 3, 2003

Related U.S. Application Data
(63) Continuation of application No. PCT/EP02/04808, filed on May 2, 2002.

(30) Foreign Application Priority Data
May 2, 2001 (DE) 101 21 471.5

(51) Int. Cl. A61K 9/70

(52) U.S. Cl. 424/449

(57) ABSTRACT

The invention is a self-adhesive, surface-doped active ingredient-containing matrix patch for controlled delivery of active ingredients to the skin having an absorbent, self-adhesive matrix, where the active ingredient is applied in dissolved or liquid form to the side of the matrix intended for skin or wound contact.

### CROSS-REFERENCE TO RELATED APPLICATIONS

This is a continuation application of PCT/EP02/04808, filed May 2, 2002, which is incorporated herein by reference in its entirety, and also claims the benefit of German Priority Application No. 101 21 471.5, filed May 2, 2001.

### FIELD OF THE INVENTION

The invention relates to surface-doped active ingredient-containing patches.

### BACKGROUND OF THE INVENTION

Active ingredient-containing patches for transdermal administration have been described many times in the literature and in patents.

Transdermal patch systems can be differentiated for example according to their construction.

In the membrane-controlled transdermal therapeutic systems, a separate active ingredient reservoir is located between an outer impermeable cover layer and a semipermeable control membrane that controls the release of the active ingredient into the skin and is combined with an additional adhesive layer for fixation to the skin.

Since the individual components of these systems with a complicated construction must be carefully matched with one another, manufacture is costly.

In the matrix-controlled systems, an intrinsic active ingredient reservoir is constructed by homogeneous dispersion of the active ingredient in a polymer matrix or a gel matrix. In this case, the polymer matrix or gel matrix ideally has self-adhesive properties so that it is unnecessary to fix the matrix on the skin by additional application of an adhesive layer. In the simplest case, the active ingredient-containing matrix is located between a cover layer firmly anchored thereto and a detachable separating layer.

The active ingredient is normally blended homogeneously in the polymer matrix or gel matrix by dissolving, dispersing, suspending, extruding, kneading, mixing or similar processes, in some cases at elevated temperature.

EP 0 219 762 A1 describes a process for producing active ingredient-containing, water-soluble sheets based on starch, gelatin, glycerol and/or sorbitol and, where appropriate, natural and/or synthetic resins and gums for oral administration. In this case, roll-coating processes are used to apply active ingredient-containing aqueous coatings onto said sheets with a constant layer thickness. The purpose of the water solubility is that the sheet dissolves or swells in the gastric juice medium on later use.

EP 0 353 972 A1 describes an adhesive patch with a discontinuous acrylate- or rubber-based adhesive layer in the form of geometric patterns of dots, which may also contain active ingredients.

DE 32 02 775 A1 describes an adhesive patch produced by printing an adhesive surface with active ingredient segments in a screen printing process. Although a patch of this type can be produced economically, it has disadvantages in the storage stability owing to the possibility of the active ingredient migrating into the adhesive matrix over lengthy periods. This is important in particular for authorization of medicinal products.

To improve the storage stability of the active ingredient patch, EP 0 170 010 A1 modifies the coating by a screen printing process through the application of separate active ingredient segments and adhesive segments. In this case, the process becomes more complicated through the use of two synchronously running screen printing rolls that are matched to one another.

A similar approach to the spatial separation of active ingredient segments and adhesive segments is implemented in EP 0 170 821 A1. This describes a separating film being located between active ingredient segments and adhesive layer and preventing interaction between active ingredient and adhesive. Separating film and active ingredient are applied successively in a gravure or screen printing process. It is necessary in this case for the application parameters for distribution in area and in height to be selected carefully in order to achieve sufficient homogeneity.

In addition, the active ingredient segments in each case protrude about 160 μm above the adhesive layer. Such a separate construction means that both active ingredient and adhesive have only intermittent, i.e. discontinuous and thus in each case not full-area, skin contact since the two are always spatially separate from one another. Such an adhesive patch therefore represents, in terms of the adhesive properties and relative to the effect of the medicinal product on the skin surface, a compromise and thus is not an optimal system.

WO 89/07429 A1 describes a transdermal therapy system which consists of a multilayer laminate composed of an active ingredient-impermeable cover layer, an adhesive layer, an adsorbent layer and a further adhesive layer which serves for fixation on the skin. To produce this system, the active ingredient is applied in liquid form with the aid of a printing process to the flexible adsorbent layer, preferably a nonwoven layer, which is already combined with the first adhesive layer and the cover layer. The second adhesive layer is subsequently laminated on. The intermediate layer loaded with active ingredient serves as depot only transiently because the active ingredient migrates into the adjacent adhesive layers, which are permeable to the active ingredient until an equilibrium has been set up.

### SUMMARY OF THE INVENTION

It is an object of the present invention to provide a surface-doped active ingredient-containing matrix patch for controlled delivery of active ingredients to the skin and/or into the wound, which is self-adhesive and which can be produced economically.

The object is achieved by a matrix patch as set forth in claim 1. The dependent claims encompass advantageous variants of the subject matter of the invention.

The present invention relates to self-adhesive, surface-doped active ingredient-containing matrix patches for controlled delivery of active ingredients to the skin or into the wound having an absorbent, self-adhesive matrix, where the active ingredient is applied in dissolved or liquid form onto the side of the matrix which is intended for contact with the skin or wound, specifically in such a way that the self-adhesive properties are retained after the application.

### BRIEF DESCRIPTION OF THE DRAWINGS

FIG. 1 illustrates a preferred geometric shape of the matrix patch as used in particular for blister patches.

FIG. 2 illustrates a further preferred geometric shape of the matrix patch.

FIG. 3 illustrates a further preferred geometric shape of the matrix patch.

FIG. 4 illustrates a further preferred geometric shape of the matrix patch.

FIG. 5 illustrates a further preferred geometric shape of the wound dressing.

FIG. 6 shows three further embodiments of a matrix patch of the invention, specifically in cross section.

Detailed Description of the Preferred Embodiments

Suitable in principle for the application of the active ingredient are coating processes, in particular printing processes, such as, for example, the Accu gravure process or the flexographic printing process, also the screen printing process as a particular type of gravure printing. Contact-free spraying processes are also advantageous.

It should be particularly emphasized that on application the active ingredient is put onto the surface of an adhesive layer and penetrates in liquid or dissolved form into the polymer matrix, and is distributed therein until an equilibrium is set up and, after evaporation of any solvent which is present, remains in the polymeric support. The self-adhesive properties of the doped polymer matrix are then recovered.

In a further possible advantageous embodiment, the solvent is only partly evaporated after application of the active ingredient solution, because the residual amount of solvent remaining in the polymer matrix is advantageously able to control, i.e. further, accelerate or delay, release of the active ingredient on use of the active ingredient-containing patch product.

These processes proves to be particularly advantageous for active ingredients which do not show inert behavior toward reactive polymer systems such as polyurethane systems, and thus cannot be produced by known prior art processes.

Suitable as backing layers are absorbent, self-adhesive polymers, preferably polyurethanes, also in foamed form, which may additionally comprise fillers or auxiliaries such as absorbent materials or penetration promoters.

The active ingredient may in the case of liquid or molten active ingredients be applied in a particularly mild manner directly or using a vehicle. The vehicles used are suitable solvents for the active ingredients, such as, for example, alcohols and acetone, and water or others. The solutions may be modified with penetration enhancers or viscosity aids. The active ingredient can also be employed directly in a suitable enhancer. The printing or coating step can be repeated more than once.

A large number of substance groups are used as active ingredients, for example essential oils, cosmetic skin-care additives, pharmaceutically active substances or antiseptics.

Transdermal therapeutic systems doped with essential oils and their constituents (for example eucalyptus oil, peppermint oil, camphor or menthol) have a long-term therapeutic effect on colds, headaches and other indications.

Concentrates obtained from plants and employed as natural raw materials mainly in the perfume industry and foodstuffs industry, and consisting more or less of volatile compounds such as, for example, true essential oils, citrus oils, absolutes, resinoids, are known as essential oils.

The term is often also used for the volatile constituents still present in the plants. However, essential oils in the real sense mean mixtures of volatile components produced by steam distillation from plant raw materials.

True essential oils consist exclusively of volatile components whose boiling points are mainly between 150 and 300° C. Unlike, for example, fixed oils, they therefore do not leave permanent transparent greasy spots behind when dabbed onto filter paper. Essential oils comprise mainly hydrocarbons or monofunctional compounds such as aldehydes, alcohols, esters, ethers and ketones.

Parent compounds are mono- and sesquiterpenes, phenylpropane derivatives and long-chain aliphatic compounds.

In some essential oils, one constituent predominates (for example eugenol comprising more than 85% of clove oil), while others have extremely complex compositions. The organoleptic properties are often determined not by the main components but by subsidiary or trace constituents, such as, for example, by the 1,3,5-undecatrienes and pyrazines in galbanum oil. The number of identified components in many of the commercially significant essential oils is up in the hundreds. Very many constituents are chiral, with one enantiomer very often predominating or being exclusively present, such as, for example, (−)-menthol in peppermint oil or (−)-linalyl acetate in lavender oil.

In an advantageous embodiment, the matrix comprises from 0.1 to 20% by weight, in particular 1 to 10% by weight, of essential oils which are chosen in particular from the group of eucalyptus oil, peppermint oil, camomile oil, camphor, menthol, citrus oil, cinnamon oil, thyme oil, lavender oil, clove oil, teatree oil, cajeput oil, niaouli oil, kanuka oil, manuka oil, dwarf pine oil.

Citrus oils are essential oils obtained from the peel of citrus fruits (bergamot, grapefruit, lime, mandarin, orange, lemon), often also called agrumen oils. Citrus oils consist largely of monoterpene hydrocarbons, mainly limonene (exception: bergamot oil which contains only about 40%).

Camphor means 2-bornanone, 1,7,7-trimethylbicyclo[2.2.1]heptan-2-one, see figure below.

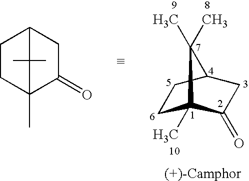

Peppermint oils are essential oils obtained by steam distillation from the leaves and flowers of various types of peppermint, occasionally also those from Mentha avensis.

Menthol has three asymmetric C atoms and accordingly exists in four diastereomeric pairs of enantiomers (cf. the formulae; the other four enantiomers are the corresponding mirror images).

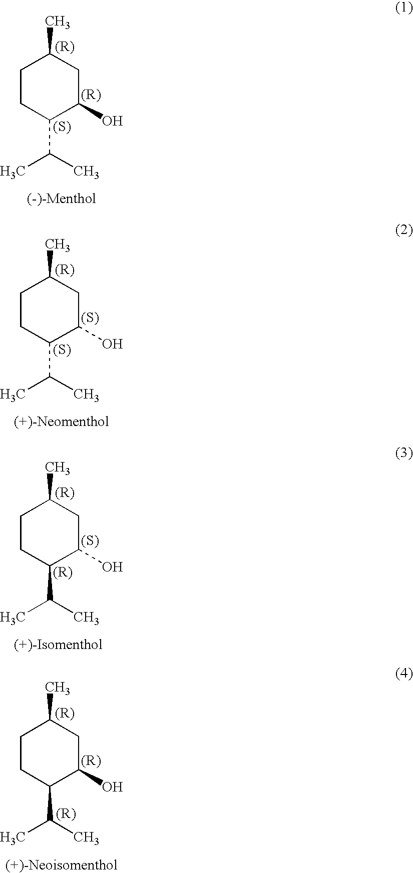

The diastereomers, which can be separated by distillation, are referred to as neoisomenthol, isomenthol, neomenthol [(+) form: constituent of Japanese peppermint oil) and menthol. The most important isomer is (−)-menthol (levomenthol), shining prisms with a strong peppermint-like odor.

When menthol is rubbed into the skin (especially on the forehead and temples), it causes a pleasant cool sensation as a result of surface anesthesia and stimulation of the cold-sensitive nerves in migraine and the like; in fact, the relevant areas show a normal or elevated temperature. These effects are not possessed by the other menthol isomers.

It is furthermore possible and advantageous to add cosmetic skin-care additives to the matrix, in particular to the extent of 0.2 to 10% by weight, very especially 0.5 to 5% by weight.

The cosmetic skin-care additives (one or more compounds) can very advantageously be chosen according to the invention from the group of lipophilic additives, especially from the following group: acetylsalicylic acid, atropine, azulene, hydrocortisone and derivatives thereof, for example hydrocortisone 17-valerate, vitamins and provitamins, for example ascorbic acid and derivatives thereof, vitamin A, vitamin A palmitate, vitamins of the B and D series, very beneficially vitamin B₁, vitamin B₁₂, vitamin D₁, provitamin B₅, pantothenic acid, but also bisabolol, unsaturated fatty acids, specifically the essential fatty acids (often also called vitamin F), especially gamma-linolenic acid, oleic acid, eicosapentaenoic acid, docosahexaenoic acid and derivatives thereof, chloramphenicol, caffeine, prostaglandins, thymol, camphor, extracts or other products of vegetable and animal origin, for example evening primrose oil, borage oil or currant seed oil, fish oils, fish liver oil or else ceramides and ceramide-like compounds and so on.

It is also advantageous to choose the additives from the group of superfatting substances, for example Purcellin oil, EUCERIT® and NEOCERIT®.

The additive(s) are furthermore particularly advantageously chosen from the group of NO synthase inhibitors, especially if the preparations of the invention are to be used for the treatment and prophylaxis of the symptoms of intrinsic and/or extrinsic skin aging, and for the treatment and prophylaxis of the harmful effects of ultraviolet radiation on the skin.

The preferred NO synthase inhibitor is nitroarginine.

It is further advantageous for the additive(s) to be selected from the group including catechins and bile acid esters of catechins and aqueous or organic extracts from plants or plant parts which have a content of catechins or bile acid esters of catechins, such as, for example, the leaves of the Theaceae family of plants, especially of the species Camellia sinensis (green tea). The typical constituents thereof (such as, for example, polyphenols or catechins, caffeine, vitamins, sugars, minerals, amino acids, lipids) are particularly advantageous.

Catechins represent a group of compounds that are to be regarded as hydrogenated flavones or anthocyanidins and represent derivatives of “catechin” (catechol, 3,3′,4′,5,7-flavanpentaol, 2-(3,4-dihydroxyphenyl)chroman-3,5,7-triol). Epicatechin ((2R,3R)-3,3′,4′,5,7-flavanpentaol) is also an advantageous additive for the purposes of the present invention.

Also advantageous are plant extracts having a content of catechins, in particular extracts of green tea, such as, for example, extracts from leaves of plants of the species Camellia spec., very particularly of the tea varieties Camellia sinenis, C. assamica, C. taliensis or C. irrawadiensis and hybrids thereof with, for example, Camellia japonica.

Preferred additives are moreover polyphenols or catechins from the group of (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called “flavones”) are advantageous additives for the purposes of the present invention. They are characterized by the following basic structure (positions of substitution indicated):

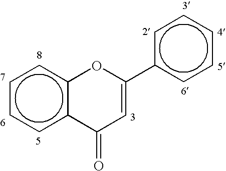

Some of the more important flavones that can also be employed preferably in preparations of the invention are listed in the following table:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kampherol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones ordinarily occur in the form of glycosides.

The flavonoids are preferably chosen according to the invention from the group of substances of the generic structural formula

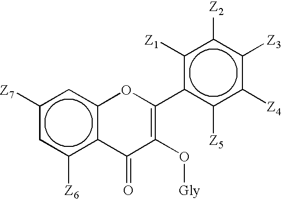

where Z₁ to Z₇ are chosen independently of one another from the group of H, OH, alkoxy- and hydroxyalkoxy-, where the alkoxy and hydroxyalkoxy groups may be branched and unbranched and have 1 to 18 C atoms, and where Gly is chosen from the group of mono- and oligoglycoside residues.

However, it is also possible according to the invention for the flavonoids advantageously to be chosen from the group of substances of the generic structural formula

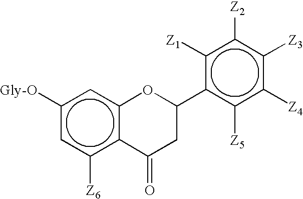

where Z₁ to Z₆ are chosen independently of one another from the group of H, OH, alkoxy- and hydroxyalkoxy-, where the alkoxy and hydroxyalkoxy groups may be branched and unbranched and have 1 to 18 C atoms, and where Gly is chosen from the group of mono- and oligoglycoside residues.

Such structures can preferably be chosen from the group of substances of the generic structural formula

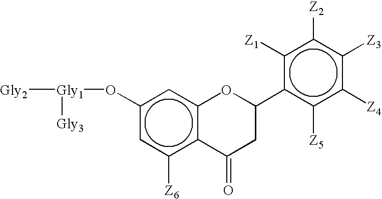

where Gly₁, Gly₂ and Gly₃ represent, independently of one another, monoglycoside residues. Gly₂ and Gly₃ may also represent, singly or together, saturations by hydrogen atoms.

Gly₁, Gly₂ and Gly₃ are preferably chosen independently of one another from the group of hexosyl radicals, in particular of the rhamnosyl radicals and glucosyl radicals. However, other hexosyl radicals, for example allosyl, altrosyl, galactosyl, gulosyl, idosyl, mannosyl and talosyl, can also be used advantageously where appropriate. It may also be advantageous according to the invention to use pentosyl radicals.

Z₁ to Z₅ are advantageously chosen independently of one another from the group H, OH, methoxy-, ethoxy- and 2-hydroxyethoxy-, and the flavone glycosides have the structure

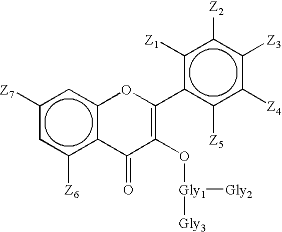

The flavone glycosides of the invention are particularly advantageously [lacuna] from the group represented by the following structure:

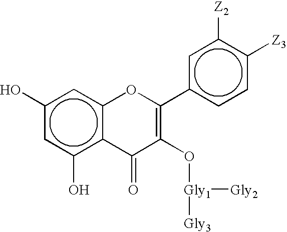

where Gly₁, Gly₂ and Gly₃ represent, independently of one another, monoglycoside residues. Gly₂ and Gly₃ may also represent, singly or together, saturations by hydrogen atoms.

Gly₁, Gly₂ and Gly₃ are preferably chosen independently of one another from the group of hexosyl radicals, in particular of the rhamnosyl radicals and glucosyl radicals. However, other hexosyl radicals, for example allosyl, altrosyl, galactosyl, gulosyl, idosyl, mannosyl and talosyl, can also be used advantageously where appropriate. It may also be advantageous according to the invention to use pentosyl radicals.

It is particularly advantageous for the purposes of the present invention to choose the flavone glycoside(s) from the group of α-glucosylrutin, α-glucosylmyricetin, α-glucosylisoquercitrin, α-glucosylisoquercetin and α-glucosylquercitrin.

Particularly preferred according to the invention is α-glucosylrutin.

Also advantageous according to the invention are naringin (aurantiin, naringenin 7-rhamnoglucoside), hesperidin (3′,5,7-trihydroxy-4′-methoxyflavanone 7-rutinoside, hesperidoside, hesperetin 7-O-rutinoside), rutin (3,3′,4′,5,7-pentahydroxyflyvone 3-rutinoside, quercetin 3-rutinoside, sophorin, birutan, rutabion, taurutin, phytomelin, melin), troxerutin (3,5-dihydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxyflavanone), eriodictyol 7-glucoside (3′,4′,5,7-tetrahydroxyflavanone 7-glucoside), flavanomarein (3′,4′,7,8-tetrahydroxyflavanone 7-glucoside) and isoquercetin (3,3′,4′,5,7-pentahydroxyflavanone-3-β-D-glucopyranoside).

It is also advantageous to choose the additive(s) from the group of ubiquinones and plastoquinones.

Ubiquinones are distinguished by the structural formula

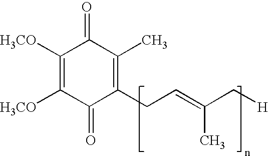

and represent the most widespread and thus best investigated bioquinones. Ubiquinones are referred to as Q-1, Q-2, Q-3, etc. depending on the number of isoprene units linked in the side chain, or as U-5, U-10, U-15, etc. according to the number of C atoms. They occur preferentially with particular chain lengths, for example with n=6 in some microorganisms and yeasts. Q10 predominates in most mammals, including humans.

Coenzyme Q10 is particularly advantageous, and is characterized by the following structural formula:

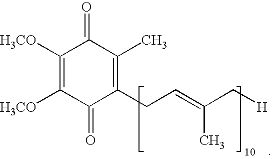

Plastoquinones have the general structural formula

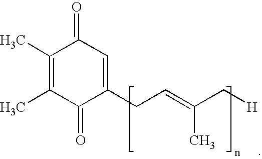

Plastoquinones differ in the number n of isoprene residues and are designated correspondingly, for example PQ-9 (n=9). Other plastoquinones with different substituents on the quinone ring also exist.

Creatine and/or creatine derivatives are also preferred additives for the purposes of the present invention. Creatine is distinguished by the following structure:

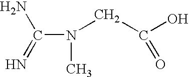

Preferred derivatives are creatine phosphate and creatine sulfate, creatine acetate, creatine ascorbate and the derivatives esterified with mono- or polyfunctional alcohols on the carboxyl group.

A further advantageous additive is L-carnitine [3-hydroxy-4-(trimethylammonio)butyric acid betaine]. Acyl-carnitines which [lacuna] chosen from the group of substances of the following general structural formula

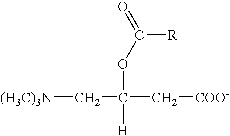

where R is chosen from the group of branched and unbranched alkyl radicals having up to 10 carbon atoms are also advantageous additives for the purposes of the present invention. Propionyl carnitine and, in particular, acetyl carnitine are preferred. Both enantiomers (D and L forms) can advantageously be used for the purposes of the present invention. It may also be advantageous to use any mixtures of enantiomers, for example a racemate of D and L forms.

Further advantageous additives are sericoside, pyridoxol, vitamin K, biotin and aromatizing substances.

The list of additives and additive combinations mentioned as being usable in the preparations of the invention is, of course, not intended to be limiting. The additives can be used single or in any combinations with one another.

It is then possible to add pharmaceutically active substances to the matrix of the active ingredient-containing matrix patch, preferably up to 40% by weight, in particular 0.01 to 25% by weight, very particularly 0.1 to 10% by weight.

Typical active ingredients are—without claiming completeness within the scope of the present invention:

| Indication: | Active ingredient |
|---|---|
| Antimycotics | Nafitine |
| | Amorrolfine |
| | Tolnaftate |
| | Ciclopirox |
| | Clotrimazole |
| Antiseptics | Thymol |
| | Eugenol |
| | Triclosan |
| | Hexachlorophene |
| | Benzalkonium chloride |
| | Clioquinol |
| | Quinolinol |
| | Undecenoic acid |
| | Ethacridine |
| | Chlorhexidine |
| | Hexetidine |
| | Dodicin |
| | Iodine |
| Non-steroidal anti-inflammatory | Glycol salicylate |
| drugs | Flufenamic acid |
| | Ibuprofen (or salts) |
| | Etofenamate |
| | Ketoprofen |
| | Piroxicam |
| | Indomethacin |
| | Diclofenac (or salts) |
| | Lidocaine (or salts) |
| Antipruritics | Polidocanol |
| | Isoprenaline |
| | Crotamiton |
| Local anesthetics | Benzocaine |
| Antipsoriatics | Ammonium |
| | bitumasulfonate |
| Keratolytics | Urea |
| | Salicylic acid (or salts) |

Further active ingredients which promote wound healing, such as dexpanthenol (panthenol) or the corresponding D and L isomers or silver sulfadiazine, can likewise be employed.

The skilled worker knows that dexpanthenol is the international nonproprietary name for D-(+)-2,4-dihydroxy-N-(3-hydroxypropyl)-3,3-dimethylbutyramide (panthenol) which has wound-healing action and the formula

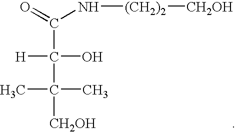

Dexpanthenol is used as active ingredient in a particularly advantageous embodiment of the matrix patch, very particularly in dissolved form (for example water or alcohols).

It is also possible to employ D-Panthenol 75 L (Roche) directly. This form of D-panthenol is supplied in solution in water (75%), stabilized with L-lactone, viscosity 541 cP at 25° C., pharmaceutical quality.

The amount of dexpanthenol applied per matrix patch is, in particular, 5 to 10 000 μg per cm², preferably 50 to 1 000 μg per cm². This corresponds to a dexpanthenol content in the matrix of 0.01 to 20%, preferably 0.1 to 2%, when the weight per unit area of the polyurethane backing which is preferably employed is 500 g/m²=50 mg/cm².

It is additionally possible to mention also hyperemic active ingredients such as natural active ingredients from cayenne pepper, capsicum extracts or synthetic active ingredients such as nonivamide, nicotinic acid derivatives, preferably bencyl nicotinate or propyl nicotinate, and anti-inflammatory agents and/or analgesics.

Examples which may be mentioned are capsaicin

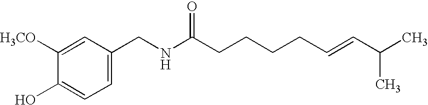

### [8-Methyl-trans-6-nonenoic acid (4-hydroxy-3-methoxybenzylamide)]Nonivamide

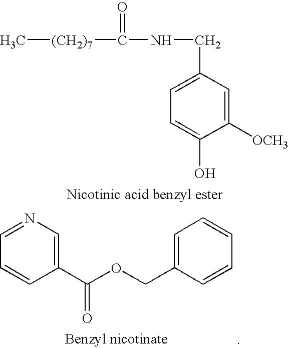

Active ingredients which should be emphasized as particularly important are the disinfectants and antiseptics, so that use thereof in the matrix is to be stressed once again.

Substances designated as disinfectants are those suitable for disinfection, i.e. for controlling pathogenic microorganisms (for example bacteria, viruses, spores, microfungi and molds), in particular generally by use on the surface of the skin, clothing, equipment, wounds, but also drinking water, foodstuffs, seeds (dressing) and as soil disinfectant.

Disinfectants particularly for local use, for example for disinfecting wounds, are also referred to as antiseptics.

The subject matter of the invention proves to be particularly advantageous in the case of active ingredients having chemically reactive functionalities such as, for example, carboxyl or hydroxyl groups, which may adversely affect and interfere with the reaction in doping processes otherwise customary by mixing the active ingredients with the components of the reactive polyurethane system. These active ingredients include, for example, ibuprofen, lidocaine, salicylic acid, ascorbic acid, dexpanthenol, menthol and aqueous or alcoholic extracts of camomile, hamamelis, marigold, peppermint oil or other essential oils.

In a further advantageous embodiment of the matrix, superabsorbents are added thereto.

The matrix then, in a further advantageous embodiment, comprises an in particular hydrophilic filler based on cellulose and derivatives thereof, the average particle size of which is in the range from 20 to 60 μm, since it has surprisingly been found in the selection of the fillers that, in particular, fillers based on silicon dioxide or cellulose are suitable, the latter having an isotropic morphology and not being prone to swelling on contact with water. Fillers particularly suitable in this connection are those having a particle size of less than or equal to 100 μm.

The use of hydrophilic fillers in a nonpolar matrix is known in the literature. They are described in EP 0 186 019 A1 explicitly for use in transdermal therapeutic systems. However, in this case only at a concentration of 3 to 30% by weight, without details of these fillers being mentioned. Experience shows that systems with a filler content of more than 30% by weight show a marked loss of tack and become hard and brittle. They thereby lose the fundamental requirement of a transdermal therapeutic system.

Fillers based on microcrystalline or amorphous cellulose are preferably employed in considerably higher concentrations without adversely affecting the adhesive properties, especially if they have an isotropic morphology with a particle size not exceeding 100 μm. Higher filler contents are desirable to improve the wearing properties, especially on long-lasting and repeated use.

Also preferably added to the matrix are permeation-promoting constituents in the concentration range up to 30% by weight, preferably from 5 to 15% by weight.

These include, for example lipophilic solubilizers/enhancers such as decyl oleate, isopropyl myristate and isopropyl palmitate (IPM and IPP), 2-octyldodecanol etc.

The matrix particularly advantageously consists of polyurethane.

DE 196 18 825 A1 relates to suitable polyurethanes and discloses hydrophilic, self-adhesive polyurethane gels which consist of

a) polyetherpolyols having 2 to 6 hydroxyl groups and OH numbers of from 20 to 112 and an ethylene oxide (EO) content of ≧10% by weight,

b) antioxidants,

c) bismuth(III) carboxylates based on carboxylic acids having 2 to 18 C atoms and soluble in the polyols a), as catalysts, and

d) hexamethylene diisocyanate,

with a product of the functionalities of the polyurethane-forming components a) and d) of at least 5.2, where the amount of catalyst c) is from 0.005 to 0.25% by weight based on the polyol a), the amount of antioxidants b) is in the range from 0.1 to 1.0% by weight based on polyol a), and a ratio of free NCO groups of component d) to free OH groups of component a) (isocyanate index) is chosen in the range from 0.30 to 0.70.

Polyetherpolyols preferably having 3 to 4, very particularly preferably 4, hydroxyl groups and having an OH number in the range from 20 to 112, preferably 30 to 56, are employed. The ethylene oxide content in the polyetherpolyols employed according to the invention is preferably >20% by weight.

The polyetherpolyols are known as such per se and are prepared by self-polymerization of epoxides such as ethylene oxide, propylene oxide, butylene oxide or tetrahydrofuran, or by addition of these epoxides, preferably of ethylene oxide and propylene oxide—where appropriate mixed together or separately and consecutively—onto starter components having at least two reactive hydrogen atoms, such as water, ethylene glycol, propylene glycol, diethylene glycol, dipropylene glycol, glycerol, trimethylolpropane, pentaerythritol, sorbitol or sucrose. Representatives of the high molecular weight polyhydroxy compounds mentioned for use are listed for example in High Polymers, Vol. XVI, “Polyurethanes, Chemistry and Technology” (Saunders-Frisch, Interscience Publishers, New York, Vol. 1, 1962, pages 32-42).

The isocyanate component employed is monomeric or trimerized hexamethylene diisocyanate, or hexamethylene diisocyanate which has been modified by biuret, uretdione, allophanate groups or by prepolymerization with polyetherpolyols or mixtures of polyetherpolyols based on known starter components having 2 or >2 reactive H atoms and epoxides such as ethylene oxide or propylene oxide of an OH number of ≦850, preferably 100 to 600. The use of modified hexamethylene diisocyanate is preferred, in particular hexamethylene diisocyanate modified by prepolymerization with polyetherdiols of OH number 200 to 600. It is very particularly preferred for the hexamethylene diisocyanate to be modified with polyetherdiols of OH number 200-600 whose residual content of monomeric hexamethylene diisocyanate is below 0.5% by weight.

Suitable catalysts for the polyurethane gels of the invention are bismuth(III) carboxylates which are based on linear, branched, saturated or unsaturated carboxylic acids having 2 to 18, preferably 6 to 18, C atoms and which are soluble in the anhydrous polyetherpolyols a). Bi(III) salts of branched saturated carboxylic acids having tertiary carboxyl groups, such as 2,2-dimethyloctanoic acid (for example versatic acids, Shell), are preferred. Preparations of these Bi(III) salts in excess proportions of these carboxylic acids are very suitable. A solution of 1 mol of the Bi(III) salt of versatic 10 acid (2,2-dimethyloctanoic acid) in an excess of 3 mol of this acid with a Bi content of about 17% has proved outstandingly suitable.

The catalysts are preferably employed in amounts of from 0.03 to 0.1% by weight based on the polyol a).

Antioxidants suitable for the polyurethane gels of the invention are, in particular, sterically hindered phenolic stabilizers such as BHT (2,6-di-tert-butyl-4-methylphenol), Vulkanox BKF (2.2 min-methylenebis(6-tert-butyl-4-methylphenol) (Bayer AG), Irganox 1010 (pentaerythrityl tetrakis[3-(3,5-ditert-butyl-4-hydroxyphenyl)propionate]), Irganox 1076 (octadecyl 3-(3,5-ditert-butyl-4-hydroxyphenyl)propionate) (Ciba-Geigy) or tocopherol (vitamin E). Those of the α-tocopherol type are preferably employed.

The antioxidants are preferably employed in amounts of from 0.15 to 0.5% by weight based on the polyol (a).

The isocyanate index (ratio of the free NCO groups employed in the reaction to the free OH groups) of the polyurethane gel compositions of the invention is, depending on the functionality of the isocyanate and polyol components employed, in the range from 0.30 to 0.70, preferably in the range from 0.45 to 0.60. The isocyanate index necessary for gel formation can be estimated very simply from the following formula:

f_{(polyol)}·(f_{(isocyanate)}−1)·index≈2

$$Index \approx \frac{2}{{f}_{( polyol )} \cdot ( {f}_{( isocyanate )} - 1 )}$$

wherein f is the functionality of the isocyanate or polyol component

The isocyanate index actually to be used may vary by up to +20% from the calculated value depending on the desired tack or elasticity of the gel. The polyurethane gel compositions of the invention are prepared by conventional processes as described, for example, in Becker/Braun, Kunststoff-Handbuch, Vol. 7, polyurethane, pages 121 et seq., Carl-Hauser, 1983.

Further polyurethanes which are preferably employed are those disclosed in EP 0 665 856 B1.

The hydrophilic polyurethane gel foams are obtainable according to this from

1. a polyurethane gel which comprises

(A) 25-62% by weight, preferably 30-60% by weight, particularly preferably 40-57% by weight, based on the total of (A) and (B), of a covalently crosslinked polyurethane as high molecular weight matrix, and

(B) 75-38% by weight, preferably 70-40% by weight, particularly preferably 60-43% by weight, based on the total of (A) and (B), of one or more polyhydroxyl compounds which are firmly bound in the matrix by secondary valence forces and have an average molecular weight between 1000 and 12000, preferably between 1500 and 8000, particularly preferably between 2000 and 6000, and an average OH number between 20 and 112, preferably between 25 and 84, particularly preferably between 28 and 56, as liquid dispersant, the dispersant being essentially free of hydroxyl compounds with a molecular weight below 800, preferably below 1000, particularly preferably below 1500, and, where appropriate,

(C) 0-100% by weight, based on the total of (A) and (B), of fillers and/or additives, and which is obtainable by reacting a mixture of

a) one or more polyisocyanates,

b) one or more polyhydroxyl compounds with an average molecular weight between 1000 and 12000, and with an average OH number between 20 and 112,

c) where appropriate catalysts or accelerators for the reaction between isocyanate groups and hydroxyl groups and, where appropriate,

d) fillers and additives known per se from polyurethane chemistry, this mixture being essentially free of hydroxyl compounds with a molecular weight below 800, the average functionality of the polyisocyanates (F_{I}) being between 2 and 4, the average functionality of the polyhydroxyl compound (F_{P}) being between 3 and 6, and the isocyanate index (K) being given by the formula

$$K = \frac{300 \pm X}{( {F}_{I} \cdot {F}_{P} ) - 1} + 7$$

in which X≦120, preferably X≦100, particularly preferably X≦90, and the index K has values between 15 and 70, where the stated averages of molecular weight and OH number are to be understood as number averages,

2. a water-absorbing material, and

3. a non-aqueous foaming agent.

The polyurethane gels can be prepared from the starting compounds known in polyurethane chemistry by processes known per se, as described for example in DE 31 03 499 A1, DE 31 03 500 A1 and EP 0 147 588 A1. However, it is essential that the above-defined conditions are complied with in the selection of the gel-forming components because, otherwise, tack-free, elastic gels are obtained in place of self-adhesive gels.

Preferred polyhydroxy compounds are polyetherpolyols like those mentioned in detail in the abovementioned publications.

Both (cyclo)aliphatic and aromatic isocyanates are suitable as polyisocyanate components. Preferred (cyclo)aliphatic polyisocyanates are 1,6-hexamethylene diisocyanate and its biurets and trimers, and hydrogenated diphenylmethane diisocyanate (“MDI”) types. Preferred aromatic polyisocyanates are those obtained by distillation, such as MDI mixtures of 4,4′ and 2,4′ isomers or 4,4′-MDI, and tolylene diisocyanates (“TDI”) types.

The diisocyanates may be chosen in particular for example from the group of unmodified aromatic or aliphatic diisocyanates or else from modified products formed by prepolymerization with amines, polyols or polyetherpolyols.

The polyurethane composition may be unfoamed, foamed, unfilled or employed with additional fillers such as, for example, superabsorbents, titanium dioxide, zinc oxide, plasticizers, dyes etc. It is additionally possible to use hydrogels in semisolid to solid form with active constituents for the central zone.

The polyurethane gels may, where appropriate, comprise additives known per se from polyurethane chemistry, such as, for example, inorganic- or organic-based fillers and short fibers, metal pigments, surface-active substances or liquid extenders such as substances having a boiling point above 150° C.

Examples of organic fillers which may be mentioned are barytes, chalk, gypsum, kieserite, soda, titanium dioxide, cerium oxide, quartz sand, kaolin, carbon black and hollow microspheres.

Organic fillers which can be employed are, for example, powders based on polystyrene, polyvinyl chloride, urea-formaldehyde and polyhydrazodicarbonamide. Suitable short fibers are, for example, glass fibers with a length of 0.1-1 mm or fibers of organic origin, such as, for example, polyester or polyamide fibers. Metal powders such as, for example, iron or copper powder can likewise also be used in the gel formation. In order to confer the desired color on the gels, the organic- or inorganic-based dyes or color pigments known per se for the coloring of polyurethanes can be used, such as, for example, iron oxide or chromium oxide pigments, phthalocyanine- or monoazo-based pigments. Examples of surface-active substances which may be mentioned are cellulose powder, activated carbon and silica products.

The adhesive properties of the gels can be modified by adding where appropriate additions of polymeric vinyl compounds, polyacrylates and other copolymers customary in adhesives technology, or else adhesives based on natural substances up to a content of 10% by weight based on the weight of the gel composition.

Preferred water-absorbing materials are the water-absorbing salts, known as superabsorbents, of polyacrylates and copolymers thereof, especially the sodium or potassium salts. They may be uncrosslinked or crosslinked and are also available as commercial products. Particularly suitable products are those disclosed in DE 37 13 601 A1, as well as superabsorbents of the new generation having only small contents of water removable by drying and high swelling capacity under pressure.

Preferred products are slightly crosslinked polymers based on acrylic acid/sodium acrylate. Such sodium polyacrylates are obtainable as Favor T (Chemische Fabrik Stockhausen GmbH, Germany). Further absorbents, for example carboxymethylcellulose and karaya, are likewise suitable.

The degree of foaming can be varied within wide limits by the incorporated amounts of foaming agent.

It is further preferred for the matrix to have a thickness of from 10 to 3 000 μm, very particularly 30 to 1 000 μm.

The matrix may additionally have on the side facing away from the skin or wound a covering of a backing material, for example consisting of sheets (for example of PU, PE or PP), nonwovens, wovens, foams, metallized sheets, composite materials, cotton etc.

A metallocene polyethylene nonwoven is suitable, for example.

The metallocene polyethylene nonwoven preferably has the following properties:

a basis weight of from 40 to 200 g/m², in particular from 60 to 120 g/m², and/or

a thickness of from 0.1 to 0.6 mm, in particular from 0.2 to 0.5 and/or

a lengthwise ultimate tensile stress elongation of from 400 to 700% and/or

a transverse ultimate tensile stress elongation of from 250 to 550%.

It is thus possible to employ as backing materials known webs which are mechanically consolidated, either by overstitching with separate yarns or by interlooping.

In the first case, the resulting structures are web-yarn stitch bonds. They are produced from a fiber web, which may be, for example, of cross-plated configuration, by overstitching with separate yarns in pillar stitch formation or tricot formation. These webs are known under the name “Maliwatt” (from Malimo) or Arachne.

The second type of consolidation likewise preferably starts from a cross-plated web. During the consolidation operation, needles draw out fibers from the web itself and form them into loops, with stitches being formed in pillar stitch formation. This web stitch bond is widely used under the name “Maliviies”, likewise from Malimo.

A review of the various types of mechanically consolidated fiber nonwovens can be found in the article “Kaschierung von Autopolsterstoffen mit Faserviiesen” by G. Schmidt, Melliand Textilberichte 6/1992, pages 479 to 486.

It can be stated in summary that suitable backing materials are all rigid and elastic sheet-like structures made from synthetic and natural raw materials. Preferred backing materials can be employed in such a way that they comply with the properties of a functionally appropriate dressing. Textiles mentioned by way of example are those such as wovens, knits, lays, nonwovens, laminates, nets, sheets, foams and papers. In addition, these materials may be pretreated and/or aftertreated. Common pretreatments are corona and hydrophobization; customary aftertreatments are calendering, thermal conditioning, laminating, punching and sheathing.

A further possibility is for a layer of adhesive composition, in particular based on PU, acrylates or rubber, to be present between the matrix and the backing material.

Finally, the matrix and/or the backing material coated with the adhesive composition may, if the matrix is not present on the whole area of the backing material, be covered with the usual release paper.

The matrix patch of the invention may have any desired shape, with preference for a regular shape such as rectangular, square, circular or oval.

Preferred embodiments of the subject matter of the invention, and several figures, may be described by way of example below, without thereby wishing unnecessarily to restrict the invention.

### EXAMPLES

Various polyurethane backings were covered with active ingredient solutions.

### Preparation of the Active Ingredient Solutions

### Active Ingredient Solution 1

100 g of Dexpanthenol 75 L (Roche) and 900 g of ethanol were mixed with stirring. The solution contains 7.5% by weight dexpanthenol.

### Active Ingredient Solution 2

220 g of ibuprofen are dissolved in 728 g of ethanol by stirring. After a clear solution is obtained, 52 g of water are mixed in. The solution contains 22% by weight ibuprofen.

### Active Ingredient Solution 3

220 g of salicylic acid are dissolved in 880 g of ethanol by stirring. The solution contains 22% by weight salicylic acid.

### Active Ingredient Solution 4

57 g of ibuprofen and 18 g of Carbopol 941 (BF Goodrich) are dissolved in 925 g of ethanol by stirring. The solution contains 5.7% by weight ibuprofen. The viscosity is 4 dPas.

### Active Ingredient Solution 5

135 g of salicylic acid and 8 g of Carbopol 941 are dissolved in 857 g of ethanol by stirring. The solution contains 13.5% by weight salicylic acid. The viscosity is 2.5 dPas.

### Active Ingredient Solution 6

124 g of nonivamide and 34 g of Carbopol 941 are dissolved in 631.5 g of ethanol and 210.5 g of water by stirring. The solution contains 12.4% by weight ibuprofen. The viscosity is 3 dPas.

### Production of Active Ingredient-Containing Patches

### Examples 1 to 6

Unfoamed and foamed polyurethane backings were coated, after removal of the release paper, on the adhesive side with active ingredient solution 1 to 6. After passing through a drying channel, the coating side was covered with a release paper or release sheet and wound up to give a bundle. The web which had been printed one or more times was divided into individual patches of any desired size and sealed in a diffusion-tight primary packaging, for example in a PE/aluminum/paper composite material.

Table 1 shows the results of the analyses on examples 1 to 6.

TABLE 1
Homogeneity of active ingredient distribution

| | | Active | Average actual | |
|---|---|---|---|---|
| Example | Backing | ingredient | content | rel. SD |
| Example 1 | polyurethane | DEX | 493 μg/cm² | 3.8% (n = 30) |
| Example 2 | polyurethane | IBU | 553 μg/cm² | 5.2% (n = 15) |
| Example 3 | polyurethane | NVA | 633 μg/cm² | 4.5% (n = 15) |
| Example 4 | polyurethane | NVA | 227 μg/cm² | 3.7% (n = 15) |
| | foam | | | |
| Example 5 | polyurethane | IBU | 69.9 μg/cm² | 5.7% (n = 15) |
| | foam | | | |
| Example 6 | polyurethane | SAS | 102 μg/cm² | 3.7% (n = 15) |
| | foam | | | |

### Example 7

### Whole-Area Coating

A polyurethane layer applied at a rate of 380 g/m² was produced. A 100 μm manual knife coater was used to produce a multilayer polyurethane coating. Drying to constant weight took place between the coating steps. After the coating, the bond strength to steel was determined by comparison with the uncoated sample. The results are shown in table 4. After the coating and subsequent drying, the bond strength returned to the original level.

TABLE 2

| | Polyurethane, 380 g/m² | | |
|---|---|---|---|
| | Uncoated | 3 coats | 4 coats |
| Theoretical. amount | 0 | 2 mg/cm² IBU | 3 mg/cm² IBU |
| of active | | | |
| ingredient | | | |
| Bond strength to | 1.81 N/cm | 1.91 N/cm | 1.83 N/cm |
| steel 90° | | | |
| rel. SD (n = 13) | 0.23 N/cm | 0.13 N/cm | 0.15 N/cm |

### Example 8

### Active Ingredient Release

The release of dexpanthenol was investigated on a sample produced by the process corresponding to example 1. The product had a dexpanthenol content of 20.6%.

A buffered physiological saline solution (PBS pH=7.2) was passed as release medium over 5 samples each with an area of 3.8 cm². The dexpanthenol concentration in the medium was determined by HPLC and, from this, the relative release based on the initial content of the samples was determined.

The release after 5 h was 94.5%, with a relative standard deviation of 5.4%.

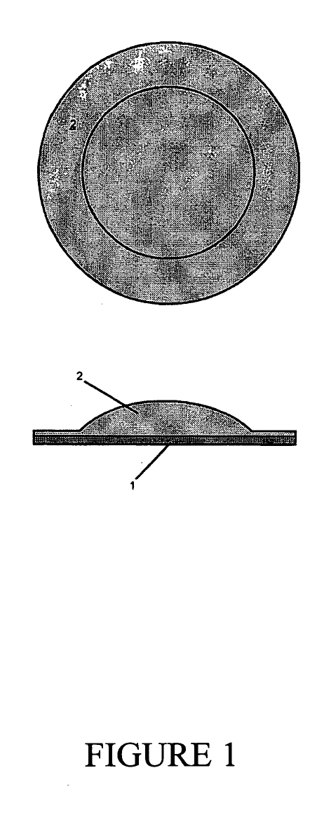

FIG. 1 illustrates a preferred geometric shape of the matrix patch as used in particular for blister patches.

The patch has a circular shape (diameter 100 mm), consisting of a polyurethane matrix 2 that slopes down toward the edge. The polyurethane matrix 2 initially slopes down uniformly and terminates in a 20 mm-wide ring where the thickness remains constant. The polyurethane matrix 2 has a substantially semiconvex shape in the middle and is accordingly comparable to a semiconvex lens.

The thickness of the polyurethane matrix 2 is 2.3 mm in the middle and 0.7 mm at the edge.

Finally, the polyurethane matrix 2 is covered by a siliconized paper 1 in order to prevent soiling or contamination of the matrix 2.

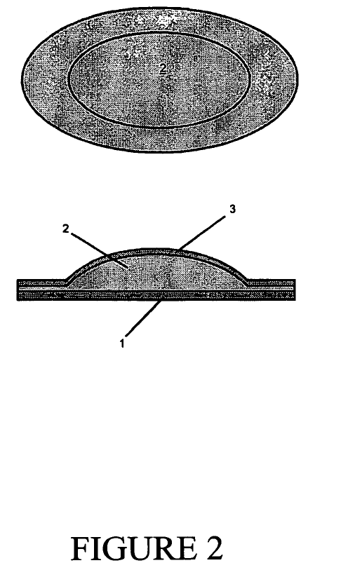

FIG. 2 illustrates a further preferred geometric shape of the matrix patch.

The patch has an ellipsoidal shape (length of the axes 42 mm and 68 mm respectively), consisting of a polyurethane matrix 2 that slopes down toward the edge. The polyurethane matrix 2 initially slopes down uniformly and terminates in a ring approximately 11 mm wide where the thickness remains constant. The polyurethane matrix 2 has an essentially semiconvex shape in the middle and is accordingly comparable to a semiconvex lens.

The PU matrix 2 is covered on the side facing away from the skin with a PE sheet 3.

The thickness of the polyurethane matrix 2 together with PE sheet 3 is 1.6 mm in the middle and 0.3 mm at the edge.

Finally, the polyurethane matrix 2 is covered with a siliconized paper 1 in order to prevent soiling or contamination of the matrix 2.

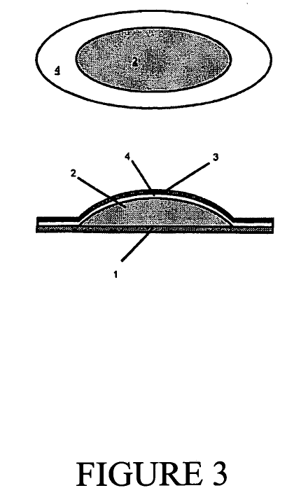

FIG. 3 illustrates a further preferred geometric shape of the matrix patch.

The patch has an ellipsoidal shape (length of the axes 110 mm and 65 mm respectively), consisting of a polyurethane matrix 2 that slopes down toward the edge. The polyurethane matrix 2 has an essentially semiconvex shape in the middle and is accordingly comparable to a semiconvex lens with a length of the axes of 72 mm and 34 mm respectively.

The PU 2 is covered on the side facing away from the skin with a PE sheet 3 that is coated over its whole area with the IPP-containing polyurethane-based adhesive layer 4. In the embodiment of the patch shown here, the entire periphery of the adhesive layer 4 is not covered by the polyurethane matrix 2. This results in two concentric zones of chemically different adhesive compositions 2, 4 that differ in terms of adherence, absorptivity and cushioning properties.

The thickness of the polyurethane matrix 2 together with PU sheet 3 and adhesive layer 4 is 1.3 mm in the middle and 0.15 mm at the edge.

Finally, the polyurethane matrix 2 is covered with a siliconized paper 1 in order to prevent soiling or contamination of the matrix 2.

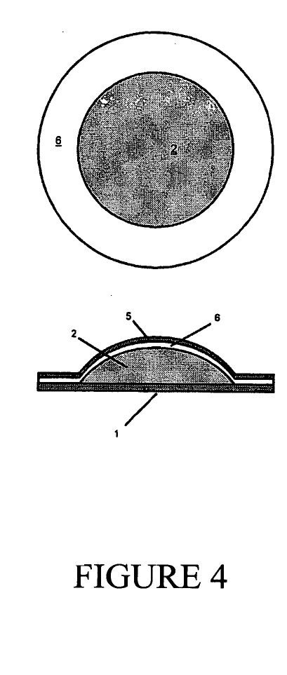

FIG. 4 illustrates a further preferred geometric shape of the matrix patch.

The patch has a circular shape (diameter 100 mm), consisting of a foamed polyurethane matrix 2 that slopes down toward the edge. The polyurethane matrix 2 has an essentially semiconvex shape and is accordingly comparable to a semiconvex lens with a diameter of 60 mm.

The PU matrix 2 is covered on the side facing away from the skin with a PU sheet 3 that is coated over its whole area with an acrylate-based adhesive layer 6. In the embodiment of the patch shown here, the entire periphery of the adhesive layer 6 is not covered by the polyurethane matrix 2. This results in two concentric zones of chemically different adhesive compositions 2, 6, which differ in terms of adherence, absorptivity and cushioning properties.

The thickness of the polyurethane matrix 2 together with PU sheet 3 and adhesive layer 6 is 1.5 mm in the middle and 0.1 mm at the edge.

Finally, the polyurethane matrix 2 is covered with a siliconized paper 1 in order to prevent soiling or contamination of the matrix 2.

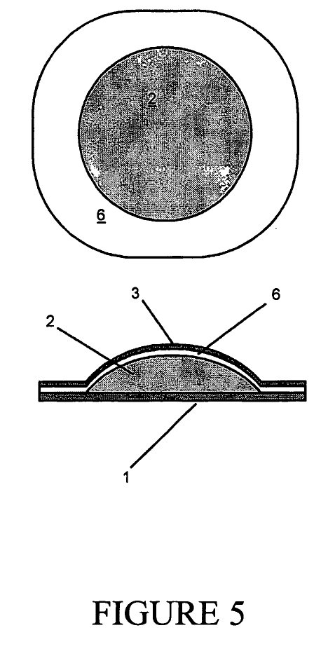

FIG. 5 illustrates a further preferred geometric shape of the wound dressing.

The patch has a square shape, with the corners of the square being rounded off (diameter of the square 50 mm), consisting of a water vapor-permeable foamed polyurethane matrix 2 that slopes down toward the edge. The polyurethane matrix 2 has an essentially semiconvex shape and is circular, and is accordingly comparable to a semiconvex lens with a diameter of 33 mm.

The PU matrix 2 is covered on the side facing away from the skin with a PU sheet 3 that is coated over its whole area with a rubber-based adhesive layer 6. In the embodiment of the patch shown here, the entire periphery of the adhesive layer 6 is not covered by the polyurethane matrix 2. This results in two concentric zones of chemically different adhesive compositions 2, 6, which differ in terms of adherence, absorptivity and cushioning properties.

The thickness of the polyurethane matrix 2 together with PU sheet 3 and adhesive layer 6 is 1.5 mm in the middle and 0.1 mm at the edge.

Finally, the polyurethane matrix 2 is covered with a siliconized paper in order to prevent swelling or contamination of the matrix 2.

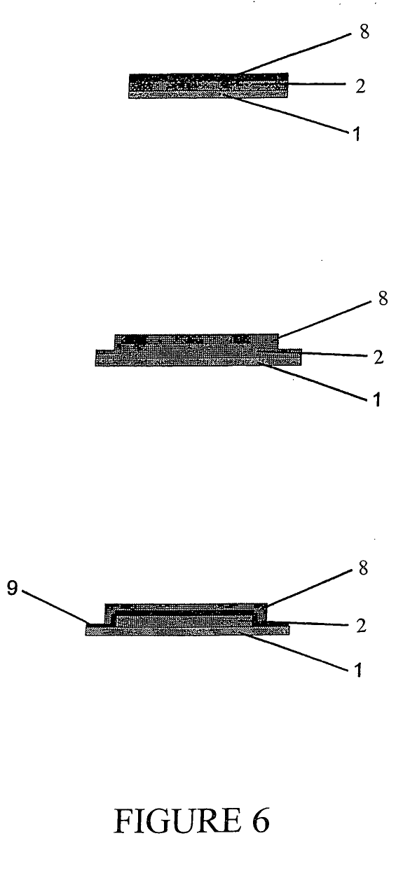

FIG. 6 shows three further embodiments of a matrix patch of the invention, specifically in cross section.

In the first embodiment of the three, the matrix patch consists of three individual layers. The doped wound pad made of the polyurethane matrix 2, is covered over its whole area with a backing material 8 on the side facing away from the wound or skin. Polymer sheets, nonwovens, wovens and combinations thereof, and sheets or textile materials made of polymers such as polyethylene, polypropylene, polyurethane or else natural fibers, are used as backing material 8.

On the side facing the wound or skin, the self-adhesive matrix 2 is covered over its whole area with a release paper 1.

In the second embodiment of the matrix patch, the matrix 2 has in the center of the patch a relatively large layer thickness, while they are thinly shaped in the edge region of the patch.

In the third embodiment, an additional adhesive coating 9 applied to the whole area of the backing material 8 is present between the matrix 2 and the backing material 8.

Unlike the matrix plasters in the first and second embodiment, in this case the matrix 2 does not extend over the entire area of the backing material 8. No matrix 2 is applied in the edge region of the backing material 8.

That Which is claimed:

1. A self-adhesive matrix patch, comprising:
an absorbent, self-adhesive matrix having a first side intended for skin or wound contact and a second side opposite the first side; and
an active ingredient applied in dissolved or liquid form to the first side of the matrix;
wherein the matrix remains adhesive on the first side even after application of the active ingredient.

2. The self-adhesive matrix patch as claimed in claim 1, wherein the active ingredient in the matrix is distributed until an equilibrium results and remains in the matrix after evaporation of any solvent that is present.

3. The self-adhesive matrix patch as claimed in claim 1, wherein the active ingredient is applied by a printing process.

4. The self-adhesive matrix patch as claimed in claim 1, wherein the active ingredient is applied by a contactless spraying process.

5. The self-adhesive matrix patch as claimed in claim 1, wherein the matrix comprises foamed or unfoamed polyurethane.

6. The self-adhesive matrix patch as claimed in claim 1, wherein at least one of essential oils, cosmetic skin-care additives, pharmaceutically active substances and antiseptics is used as an active ingredient.

7. The self-adhesive matrix patch as claimed in claim 1, wherein the matrix comprises from 0.1 to 20% by weight of an active ingredient.

8. The self-adhesive matrix patch as claimed in claim 1, wherein the matrix comprises from 2 to 10% by weight of an active ingredient.

9. The self-adhesive matrix patch as claimed in claim 1, wherein permeation-promoting constituents are added to the matrix in a concentration range from 1.0 to 30% by weight.

10. The self-adhesive matrix patch as claimed in claim 1, wherein permeation-promoting constituents are added to the matrix in a concentration range from 10 to 25% by weight.

11. The self-adhesive matrix patch as claimed in claim 1, wherein the matrix has a thickness of from 10 to 3 000 μm.

12. The self-adhesive matrix patch as claimed in claim 1, wherein the matrix has a thickness of from 30 to 1 000 μm.

13. The self-adhesive matrix patch as claimed in claim 1, wherein the matrix comprises dexpanthenol as an active ingredient.

14. The self-adhesive matrix patch as claimed in claim 13, wherein the dexpanthenol is applied in an amount of from 5 to 10 000 μg per cm² of matrix.

15. The self-adhesive matrix patch as claimed in claim 13, wherein the dexpanthenol is applied in an amount of from 50 to 1000 μg per cm² of matrix.

16. The self-adhesive matrix patch as claimed in claim 1, wherein the active ingredient is dissolved in a solvent and the dissolved active ingredient is applied to the first side of the matrix such that at least a portion of the solvent remains in the matrix.

17. A method of making a self-adhesive matrix patch, comprising the steps of:
providing an absorbent, self-adhesive matrix having a first side intended for skin or wound contact and a second side opposite the first side; and
applying an active ingredient in dissolved or liquid form to the first side of the matrix;
wherein the matrix remains adhesive on the first side even after said applying step.

18. The method as claimed in claim 17, wherein said applying step comprises applying an active ingredient dissolved in a solvent to the first side of the matrix and evaporating the solvent.

19. The method as claimed in claim 17, wherein said applying step comprises applying an active ingredient dissolved in a solvent to the first side of the matrix and evaporating none or only a portion the solvent such that at least a portion of the solvent remains in the matrix.

20. The method as claimed in claim 17, wherein the applying step comprises printing the active ingredient onto the first side of the matrix.

21. The method as claimed in claim 17, wherein the applying step comprises spraying the active ingredient onto the first side of the matrix.

22. The method as claimed in claim 17, further comprising the step of producing the matrix from foamed or unfoamed polyurethane.

23. The method as claimed in claim 17, further comprising the step of adding permeation-promoting constituents to the matrix.

24. The method as claimed in claim 17, wherein said applying step comprises applying the active ingredient in an amount to produce 2 to 10% by weight of an active ingredient in the matrix.

25. The method as claimed in claim 17, wherein said applying step comprises applying dexpanthenol as an active ingredient.
